(19) United States
(12) Patent Application Publication
(10) Pub. No.: US 2009/0209039 A1
(43) Pub. Date: Aug. 20, 2009

(54) METHOD AND APPARATUS FOR MICROFLUIDIC INJECTION

(75) Inventors: Andrea Adamo, Cambridge, MA (US); Luigi Adamo, Cambridge, MA (US); Klavs F. Jensen, Lexington, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/370,146

(22) Filed: Feb. 12, 2009

Related U.S. Application Data
(63) Continuation of application No. PCT/US2007/018204, filed on Aug. 16, 2007.
(60) Provisional application No. 60/838,303, filed on Aug. 17, 2006.

(51) Int. Cl. C12N 15/00; C12M 1/26; C12N 5/06

(52) U.S. Cl. 435/455; 435/309.1; 435/375

(57) ABSTRACT

A method and apparatus for producing a jet or droplet of liquid. An injector device may include a reservoir in fluid communication with a nozzle, and a pressure gradient may be produced in the reservoir (e.g., by a piezoelectric element in an initial direction that is transverse to the emission direction of the jet or droplet) to produce a jet of liquid from the nozzle. The jet or droplet of liquid may be introduced through a cell membrane and into the cell interior in such a way that damage to the cell membrane that would cause cell death is avoided. An electrode may be formed adjacent a fluid channel by conducting a liquid material, such as solder, from a reservoir and into an electrode portion of an electrode channel to a location adjacent the fluid channel. A passageway between the electrode channel and the fluid channel may prevent flow of the liquid electrode material into the fluid channel during electrode formation.

This application is a continuation of International Application PCT/US2007018204, filed Aug. 16, 2007, which claims the benefit of U.S. Provisional application 60/838,303, filed Aug. 17, 2007, which are hereby incorporated by reference in its entirety.

### BACKGROUND

1. Field of Invention

In some aspects, this invention relates to an apparatus and method to produce a liquid jet and/or droplet of liquid. Some applications for the jet/droplet produced include microinjection of material into cells, crystallization, nano/pico/femto droplet generation and nanoparticle synthesis. In some aspects, this invention relates to formation of an electrode for use with a channel used to conduct flow of a fluid.

2. Related Art

Microfluidics has received attention because of its potential applications in biology, chemical engineering and other fields. For example, U.S. Pat. No. 6,913,605 discloses a device for producing pulsed microfluidic jets. The fluid jet is produced by a vapor bubble that expels fluid from a chamber and through an opening. Other known arrangements can create high speed jets of fluid and nanodroplets of a solution of interest in a gaseous environment (e.g., using ink jet printer-type technology).

### SUMMARY OF INVENTION

Aspects of the invention provide a method and apparatus for producing fluid jets and/or droplets with a highly controllable volume and/or flow rate. For example, in one embodiment, a device may be capable of generating fluid jets having a speed of from about 0.0 m/sec to about 40 m/sec and a stream diameter of about 0.05 to 20 microns. The device may also be capable of creating droplets having a controlled volume in the nanoliter, picoliter or femtoliter range.

In one illustrative embodiment, a fluidic jet/droplet generator may include a reservoir of liquid and a microfabricated nozzle through which the liquid is expelled. The nozzle may be fabricated using standard photolithographic or other techniques for creating relatively small openings of 20 microns or less. The device may also include a pressure generator, such as a piezoelectric element stack and associated diaphragm, that creates a pressure pulse in the reservoir. The pressure pulse may force liquid through the nozzle to create the desired jet and/or droplet, which may be introduced into another liquid.

Aspects of the invention may have applications in various fields such as biology, chemical engineering and others. For example, material such as genetic fragments, drugs, or other, may be delivered across a cell membrane and into a cell by a controlled jet. This feature may be an important step in experimental protocols in molecular and cellular biology research, as well as be useful in gene therapy. The inventors believe that the most effective technique to allow efficient introduction into single cells of any kind of material (e.g., lipids, proteins, carbohydrates, nucleic acids, chemicals, etc.) or structure (e.g. sub-cellular organelles or microfabricated/nanofabricated structures) is microinjection. However, microinjection devices and techniques at present are expensive and extremely slow (e.g., 20 min for an experienced operator to perform an injection into one cell). In contrast, aspects of the invention provide a device and a method that enables low cost, high throughput, quantitative, automated, cellular microinjection, making use of a high speed microfluidics jet that pierces the cell, thus delivering the compound of interest into the cell in a known amount.

Aspects of the invention also have use in chemical engineering applications. For example, crystallization of compounds can be a difficult process that is sometimes achieved only after multiple trials in different crystallization conditions. Currently, the low throughput of crystallization condition screens and the difficulty in tightly controlling such conditions are holding back the field. Moreover, current crystallization protocols make use of large volumes of reagents. With certain embodiments of the invention, it is possible to deliver picoliter-sized droplets of one solution into another solution, providing a sudden, intimate contact of the reagents. The small masses involved in the microfluidics system allow very good control of the crystallization conditions, thus enhancing the repeatability of the experiments. Moreover, the small amount of reagent used decreases cost. Aspects of the invention can be used in both screening and/or production (e.g., running many systems in parallel). Molecules of interest can be of any suitable kind ranging from proteins to drugs. Small droplet generation capabilities of embodiments of the invention can allow for the synthesis of a wide range of nanoparticles.

In one aspect of the invention, a method of introducing material into a cell includes providing a cell at a position adjacent an outlet of a nozzle, providing a reservoir containing a fluid and in fluid communication with the nozzle, producing a pressure gradient in the reservoir to urge fluid in the reservoir to move toward the nozzle, and producing a jet of liquid, including the material, from the nozzle so as to introduce the liquid through the cell membrane and into the cell interior. The introduction of the liquid into the cell interior is accomplished so as to avoid damage to the cell membrane that would cause cell death. This is in contrast to other microinjection devices which are incapable of introducing material into a cell without causing significant damage to the cell membrane.

In another aspect of the invention, a fluid injection device includes a channel constructed and arranged to carry a cell along a first path, a nozzle constructed and arranged to emit a jet or droplet of liquid from an outlet and into the channel in an emission direction, a reservoir for holding liquid and in fluid communication with the nozzle, and a pressure generator, such as a piezoelectric element, adapted to create a pressure gradient in the reservoir to cause the nozzle to emit the jet or droplet of liquid from the outlet. The jet or droplet of liquid may be emitted so as to introduce the liquid through the cell membrane and into the cell interior in such a way that damage to the cell membrane that would cause cell death is avoided. In another embodiment, the jet or droplet of liquid may be emitted so as to produce an intimate contact or sudden proximity between the surface of the cell and the ejected fluid or part of its content. This process may either deliver material to the cell or achieve localization of material of interest in the immediate proximity of a specific cell.

In another aspect of the invention, a fluid injection device includes a channel constructed and arranged to carry a material along a first path, a nozzle constructed and arranged to emit a jet or droplet of fluid from an outlet of the nozzle and into the channel in an emission direction, a reservoir for holding liquid and in fluid communication with the nozzle, and a pressure generator adapted to create a pressure gradient in the reservoir to cause the nozzle to emit the jet or droplet of liquid from the outlet. The pressure generator, e.g., a piezoelectric element, may create a pressure wave in the reservoir that initially moves in a direction transverse to the emission direction.

In another aspect of the invention, a microfluidics device includes a substrate, a fluid channel formed in the substrate and constructed and arranged to conduct liquid along a flow path, and an electrode channel formed in the substrate and having at least one conductive material reservoir in communication with an electrode portion. The electrode portion of the electrode channel may be in fluid communication with the fluid channel, e.g., to allow an electrode in the electrode portion to detect electrical characteristics in the fluid channel. In one embodiment, the electrode portion may be in communication with the fluid channel via a passageway that is arranged to prevent conductive material, when in liquid form, from flowing from the electrode channel to the fluid channel, yet may be arranged to permit fluid and electrical communication between the electrode channel and the fluid channel. In accordance with this embodiment, an electrode may be formed in the electrode channel by flowing a liquid material, such as a melted solder, from the reservoir and into the electrode portion, but the passageway may prevent flow of the liquid material into the fluid channel. Thus, an electrode may be formed so as to be in communication with the fluid channel (via the passageway), yet not interfere with the flow characteristics of the fluid channel.

These and other aspects of the invention will be apparent from the following detailed description and claims.

### BRIEF DESCRIPTION OF THE DRAWINGS

FIG. 1 shows a schematic block diagram of an injection system in an illustrative embodiment;

FIG. 2 shows a front view of an injection device in an illustrative embodiment;

FIG. 3 shows a side view of the FIG. 2 embodiment;

FIG. 4 shows a top view of a microfluidics channel with associated electrode channels in an illustrative embodiment;

FIG. 5 shows a close up view of the microfluidics channel with associated electrode channels of FIG. 4; and

FIG. 6 shows a view of a microfluidics channel and associated electrode channel in another illustrative embodiment.

### DETAILED DESCRIPTION

Aspects of the invention are described below with reference to illustrative embodiments of an injection device and microfluidic device. It should be understood that aspects of the invention are not limited to the illustrative embodiments described herein, but rather may be implemented in any suitable way. In addition, aspects of the invention may be used in any suitable combination with each other and/or alone.

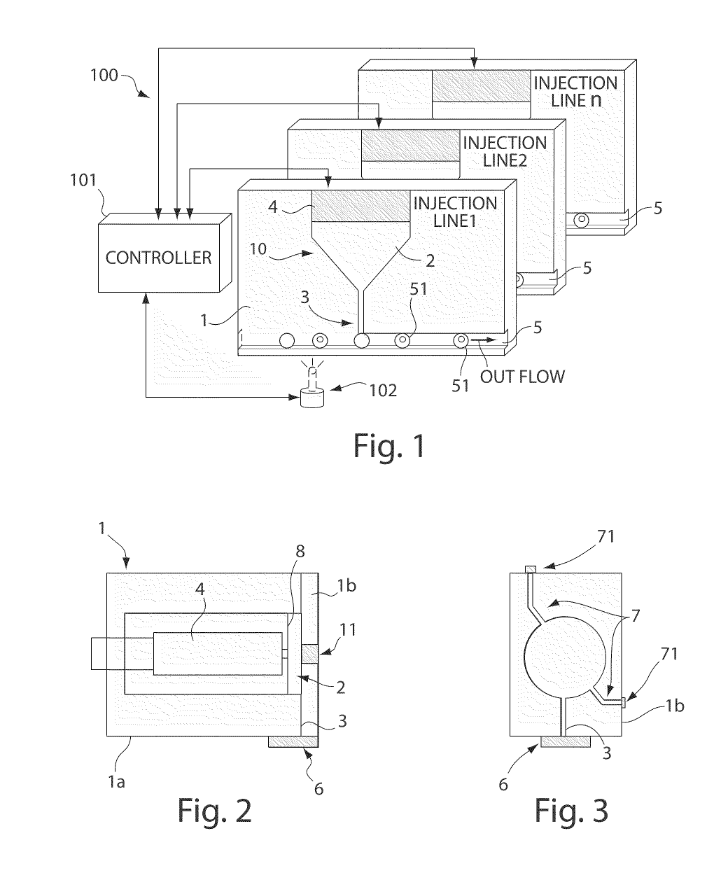

FIG. 1 shows a schematic view of an injection system that incorporates various aspects of the invention. In this embodiment, the injection system 100 is arranged to operate a plurality of injector devices 10 to introduce a jet or droplet of liquid into a respective channel 5 that is arranged to conduct the flow of a liquid past the nozzle 3 of the injector device 10. Although the channels 5 may be arranged in any suitable way, and may carry any suitable material, in this illustrative embodiment, the channels 5 have a cross-sectional size of about 15 microns×15 microns and conduct the flow of a liquid including a plurality of cells 51. (For applications in the field of crystallization or nanoparticle synthesis or droplet generation, the cross section of a channel or other arrangement used with the injection device can be on the order of several square millimeters.) The channels 5 in this embodiment are arranged so that the cells 51 are permitted to pass through the channel 5 only one at a time, i.e., so that cells 51 may be positioned adjacent the nozzle 3 in a serial fashion. The operation and arrangement of such channels 5 is well known in the art, and not described in further detail herein. However, it should be understood that aspects of the invention are not necessarily limited to the arrangement of channels 5 and/or the material contained in them.

In accordance with one aspect of the invention, the injector devices 10 may be operated to introduce a jet or droplet of liquid (e.g., where the liquid includes a marking compound, a drug, or other suitable material whether in solution, a suspended solid, or otherwise) into each of the cells 51. In this embodiment, the injector devices 10 may introduce the liquid through a membrane of the cells 51 and into the cell interior in such a way that damage to the cell membrane that would cause death of the cell 51 is avoided. It should be understood that the cells 51 need not necessarily be “living” when the liquid is introduced. Instead, the cells 51 may be dead or in another “non-living” state, yet have their cell membranes intact. Thus, introduction of liquid into a cell, whether dead or living, may be done in such a way that the cell membrane is pierced by the liquid, but damage to the cell membrane that would cause death of the cell (if living) is avoided.

This aspect of the invention is a major advance over other microfluidic jet or droplet devices, which do not have the capability of forming a jet or droplet of liquid in such a way that the liquid can penetrate a cell membrane, yet not cause damage to the membrane that would cause cell death. As discussed in more detail below, the inventors have found that a jet or droplet of liquid emitted from a nozzle 3 having a diameter under 20 microns at a speed of between about 0 m/sec to 40 m/sec and having a volume between about a femtoliter to several picoliters can be effective for introducing the liquid into a cell in a suitable way.

In this embodiment, the plurality of injection devices 10 operate under the control of a controller 101, which may include one or more general purpose computers, a network of computers, one or more microprocessors, etc. for performing data processing functions, a memory for storing data and/or operating instructions, communication buses or other communication devices for wired or wireless communication, software or other computer-executable instructions, a power supply or other power source (such as a plug for mating with an electrical outlet), relays, mechanical linkages, one or more sensors or data input devices, user data input devices (such as buttons, a touch screen or other), information display devices (such as an LCD display, indicator lights, a printer, etc.), and/or other components for providing desired input/output and control functions. The controller 101 may also control other features of the system 100, such as a pump or other device that controls flow through the channels 5 and so on.

In this embodiment, the controller 101 receives information from one or more sensors 102 regarding the presence of cells 51 in the channel 5, their speed of movement, and/or other characteristics. The sensors 102 may take any suitable form, but in this example, include one or more electrodes that provide capacitance and/or resistance information regarding local conditions in the channel 5 (which can indicate the presence/absence of a cell 51 near the sensor 102). Other sensor types that may be used include image analysis devices for imaging one or more portions of the channel 5 (e.g., using a camera or other image sensing device) and performing an analysis of the image(s), e.g., using appropriate software to locate the position and/or speed of cells 51. Another sensing approach may involve optical methods that analyze the light scatter or other optical properties of the cell 51 and surrounding fluid. In this approach, light is directed (for example, by a waveguide) inside the channel 5 at a selected location and the light scattered by the cells 51 is analyzed. This technique is currently used in some biological systems (such as fluorescence-activated cell sorting), and is often coupled with fluorescent labeling of cells by means of antibodies or cell-specific dyes. In a heterogeneous population of cells, labeling could be different for cells 51 that have different characteristics (e.g., different cells might bind different antibodies and gain different fluorescent properties). Thus, the system 100 may use this kind of labeling to allow selection of a subset of target cells to be injected within a heterogeneous population of cells provided through the channels 5. That is, the sensor 102 may identify cells 51 that should be injected with a material, and cells 51 that are not to be injected and control the injection device 10 accordingly.

Based on cell position and/or speed information, the controller 101 may control the injection devices 10 to emit a suitable jet or droplet of liquid when the cell 51 is suitably positioned relative to the nozzle 3, thereby introducing the liquid into the cell 51. The injection devices 10 may include a body 1 that has a reservoir 2 that leads to a nozzle 3. The reservoir 2 may be filled with a suitable liquid, e.g., a solution including one or more compounds such as nucleic acids (e.g. genetic fragments, RNA molecules), proteins (e.g. antibodies, in vitro synthesized peptides), lipids, carbohydrates, drug molecules, or other compounds or structures of interest. In one embodiment, the reservoir 2 may be completely filled with liquid, e.g., so there are no gas-filled voids. A pressure generator 4 may be associated with the reservoir 2 so as to introduce a pressure gradient in the reservoir 2. In this embodiment, the pressure generator 4 includes one or more piezoelectric devices that are capable of exhibiting sufficient movement to effectively change the volume of the reservoir 2 or otherwise introduce a pressure change or wave in the reservoir 2.

Upon actuation of the pressure generator 4, the fluid contained in the reservoir 2 may be pressurized (and/or a suitable pressure wave is produced) and ejected through the nozzle 3, e.g., which may include a micron-sized hole so that high speed jets can be produced. The injection device 10 may create a jet of fluid or droplet depending on the desired operation. The jet or droplet produced may be micron-sized in diameter (or other size dimension), and the volume ejected and the speed of the jet and/or droplet can be varied, e.g., by changing the length of time the pressure generator is actuated and/or how the pressure is generated in the reservoir 2. Jets produced by the injection device 10 may have speeds of between about 0 and about 40 m/sec. The ejected volume of a jet and/or droplet may be in the range from femtoliters to several picoliters or more. Jets/droplets in this size/speed/volume range have been found effective in introducing liquid into a cell without causing damage to the cell membrane that would result in cell death. For example, in one experiment, a cell was injected with a dye that fluoresces only when in contact with the cell interior (thus indicating whether the dye has been successfully introduced into the cell interior upon fluorescence of the dye). The experiment resulted in successful and stable introduction of the dye into the cell interior without alteration of the cell structure as assessed by high magnification optical microscopy. The experiment involved the use of a Hela cell suspended in 150 mM N-methyl-D-glutamine (NMDG) chloride 10 mM HEPES 10 mM Glucose (with pH adjusted to 7.4 with HCl and osmolarity adjusted to 295 mOsm). The suspended cells were caused to flow into an injection device and injected using a jet of having a speed of about 6 m/s. The injected solution was 100 micromolar of the potassium indicator PBFI (Invitrogen), dissolved in the buffer above.

Although the injection devices 10 may expel a jet or droplet into a cell 51 in a microfluidics channel 5 as shown, the devices 10 may be used to introduce liquid into any liquid or gas environment. For example, it will be understood that the injection device 10 may be used to deposit jets or droplets for other purposes, such as to deposit liquid samples into a microwell plate or other sample holder, to introduce liquid samples into a crystallization medium, etc. Moreover, the jet may be used to selectively kill cells using speeds of the jet that are sufficiently large to cause cell death, if desired.

FIGS. 2 and 3 show a front and side view, respectively, of an illustrative embodiment of an injection device 10 in accordance with the invention. In this illustrative embodiment, the injection device 10 includes a body 1 having a first part 1a and a second part 1b that are joined together, e.g., each made of aluminum, stainless steel or other suitable material(s) and attached by screws, adhesive or other fastener. A piezoelectric element 4 is mounted in the first part 1a and is separated from the reservoir 2 by a membrane 8, e.g., a sheet of flexible silicone rubber, metal or other suitable material. A pressure sensor 11 is mounted in the second part 1b and is arranged to sense the pressure in the reservoir 2, e.g., for use in control of the device 10 by the controller 101. As can be seen in FIG. 3, a pair of lines 7 communicate with the reservoir 2 to provide fluid into the reservoir 2, e.g., after it is expelled from the nozzle 3, and to allow for outflow of fluid from the reservoir 2, e.g., when flushing the reservoir 2 to remove air pockets or to prime the reservoir 2. Valves 71 can open and close the lines 7 and may communicate with a fluid source and/or a waste reservoir (not shown). For example, flow may be provided in one line 7 and out the other line 7 to ensure filling of the reservoir 2 and elimination of air or other gas from the reservoir 2. The lines 7 and nozzle 3 may be formed in the second part 1b, e.g., by machining, lithography, or any other suitable technique. Alternately, the nozzle 3 may be formed in a separate part, and then secured in place to the first and second parts 1a and 1b. This may allow for easier manufacture of the nozzle 3, which may require the formation of a small orifice, e.g., on the order of 20 microns or less.

In accordance with one aspect of the invention, the pressure generator (in this case including a piezoelectric element) creates a pressure wave or gradient that is initially oriented in a direction transverse to the direction in which the nozzle emits a jet or droplet of liquid. That is, in this illustrative embodiment, the piezoelectric element 4 operates to initially displace liquid in the reservoir 2 in a left-to-right direction as viewed in FIG. 2. However, this pressure gradient causes the nozzle 3 to emit a jet or droplet of liquid in an up-to-down direction as viewed in FIG. 2. Such an arrangement may provide advantages, such as reduced device size, reduced complexity in manufacture and/or more effective sensing of pressure characteristics in the reservoir 2, e.g., by the sensor 11. Although in this embodiment the pressure generator initially creates a pressure wave or gradient oriented in a direction perpendicular to the nozzle emission direction, the initial direction of the pressure wave may be arranged in other transverse directions between 0 and 90 degrees relative to the nozzle emission direction.

In this embodiment, the injection device 10 is associated with a plate 6 having at least one microfluidic channel (such as the channel 5 in the FIG. 1 embodiment) used to carry cells or other subjects near the nozzle 3 so that a liquid material may be introduced into the cell. Such a plate 6 may be formed of any suitable material and in any suitable way, e.g., using techniques and materials used to form microfluidic chips as are known in the art. The plate 6 may be suitably sealed to the device 10, e.g., using epoxy, so that the nozzle 3 is suitably arranged with respect to a channel 5 or other feature in the plate 6. Other kinds of adhesives or bonding techniques such as soldering or compression scaling or vacuum can be used to join the plate 6 and the device 10. Of course, it will be understood that the plate 6 may include any suitable features, such as pumps, reservoirs, valves, particle detectors, material selection features (e.g., cell diverters or other devices that can selectively sort cells from each other), and so on. Although in this embodiment the injection device 10 is made separately from the plate 6, it should be understood that the injection device 10 and plate 6, including a channel 5, may be made in an integral way, e.g., made in a same chip or other substrate. The fabrication techniques will vary according to the specific design and may include MEMS (micro electro mechanical systems) fabrication techniques. For example, portions of the injection device 10, e.g., the reservoir 2, nozzle 3, etc. may be etched or otherwise formed in a suitable substrate (such as silicon) with other components, such as the piezoelectric element, incorporated into the substrate. One or more channels 5 may also be formed in the substrate, thereby forming a single device, e.g., that may be used once for testing or other processing and then disposed.

In this illustrative embodiment, a portion of the nozzle 3 includes a terminal nozzle portion (a portion nearest the plate 6) that is formed separately from the second part 1b, and later attached to the second part 1b. To form the terminal nozzle portion in this embodiment, a micron-sized hole was etched into a silicon substrate, e.g., by standard micromachining techniques such as by deep reactive ion etching.

In this embodiment, the reservoir 2 has a diameter of about 8 mm (in other embodiments the diameter may be in the range of about 2-3 mm to about 15-20 mm or more), and a depth (dimension in the left-to-right direction of FIG. 2) of about 1 mm, but may be between about 100 micron to a few mm depending on how much fluid is to be stored for the specific experiment. Large reservoir volumes may create compliance (the liquid may be regarded as compressible for correct design), and therefore may not be desirable. The reservoir volume may range from about 0.001 ml to 1-2 ml—in this embodiment the volume is around 0.1 ml. Of course, various dimensions may be adjusted as desired.

In this embodiment, the pressure generator includes several piezoelectric elements each having a travel of about 20 microns, with external dimensions of about 18 mm thick and about 5 mm square. However, the piezoelectric element may have different dimensions and/or travel distances, e.g., 5-150 microns of travel. The membrane in this embodiment is formed by a thin metal sheet. The nozzle 3 has first a part secured in the body 1 with an internal diameter of about 500 microns and a length of a few millimeters at the end nearest the reservoir 2. The nozzle narrows in the direction toward the plate 6 to about 100 microns in diameter and a length of about 630 microns. The nozzle 3 again narrows to the terminal end with a diameter of about 4 microns and 70 microns in length at the exit side of the nozzle 3. The use of a large hole at the entrance side may have the advantage of limiting pressure drop, but is not critical, and a constant diameter or otherwise arranged through hole could also be used. Although in this embodiment, the size of the nozzle at the exit is about 4 microns, nozzles with other exit sizes, e.g., ranging from 0.05 to 20 microns, may be used in other embodiments.

When in use, the injector device 10 may create a jet with a time duration of about 1 microsecond to several milliseconds depending on the speed of the jet. Changing the speed and/or time duration of the jet may allow for adjustment of the ejected volume of the jet. The jet speed used for piercing a cell may be varied depending on cell type because different cell types may have very different mechanical behaviour.

For the construction of this illustrative embodiment, particular materials, sizes and other features have been selected for ease of fabrication. However, other materials can be used to fabricate the injection device (for instance other metals, and/or polymers, e.g., using scalable, low cost, polymer microfabrication techniques). For some embodiments, materials may be selected based on a need for chemical compatibility with the fluids that will be used in the reservoir 2, and/or sufficient mechanical stiffness to avoid dampening of the pressure wave generated by the piezoactuator or other pressure generator, and/or damage to the subject into which liquid is injected (e.g., a cell). The use of sterilizable polymers may allow development of low cost, single use sample handling systems for biological-related applications. (The piezoelectric actuator can be separated from the reservoir by a disposable, thin polymer membrane without loss of performance).

The fabrication of the device can be carried out with other methods as well. For instance, a device can be fabricated exclusively with microfabrication techniques or, as in the illustrative embodiments above, with a combination of macrofabrication (e.g., standard machine shop techniques and tools) and microfabrication techniques (e.g., photolithography, laser ablation and/or chemical etching for the micro-parts).

As mentioned above, embodiments in accordance with aspects of the invention may include other features not described above. For instance, in order to enhance the fluid handling capabilities of the microfabricated chip, valves can be included and the hydraulic design of the channels 5 in the plate 6 can be changed.

In accordance with one aspect of the invention, a plate or other substrate may include a fluid channel (such as the channel 5) to conduct liquid along a flow path, and an electrode channel in fluid and electrical communication with the fluid channel. The electrode channel may include a conductive material, such as a solder or other metal, that functions as an electrode to detect electrical characteristics in the fluid channel, e.g., a capacitance and/or resistance in the fluid channel. As discussed above, such characteristics may be exploited by a sensor 102 in detecting the presence/absence of cells 51 or other materials in a channel 5. The electrode channel may include a conductive material reservoir in communication with an electrode portion, which is the portion of the electrode channel in fluid and electrical communication with the fluid channel. In one embodiment, the electrode portion of the electrode channel may communicate with the fluid channel via a passageway that is sized so that conductive material in liquid form, e.g., melted solder, used to form the electrode does not flow through the passageway when flowing from the conductive material reservoir and into the electrode portion. Thus, a conductive electrode may be formed in the electrode channel with little/no risk of effecting the fluid flow characteristics of the fluid channel This aspect of the invention may provide for easier manufacture of an electrode that communicates with a fluid channel, in part because an effective electrode may be provided with minimized risk of damaging or otherwise affecting flow in the channel 5.

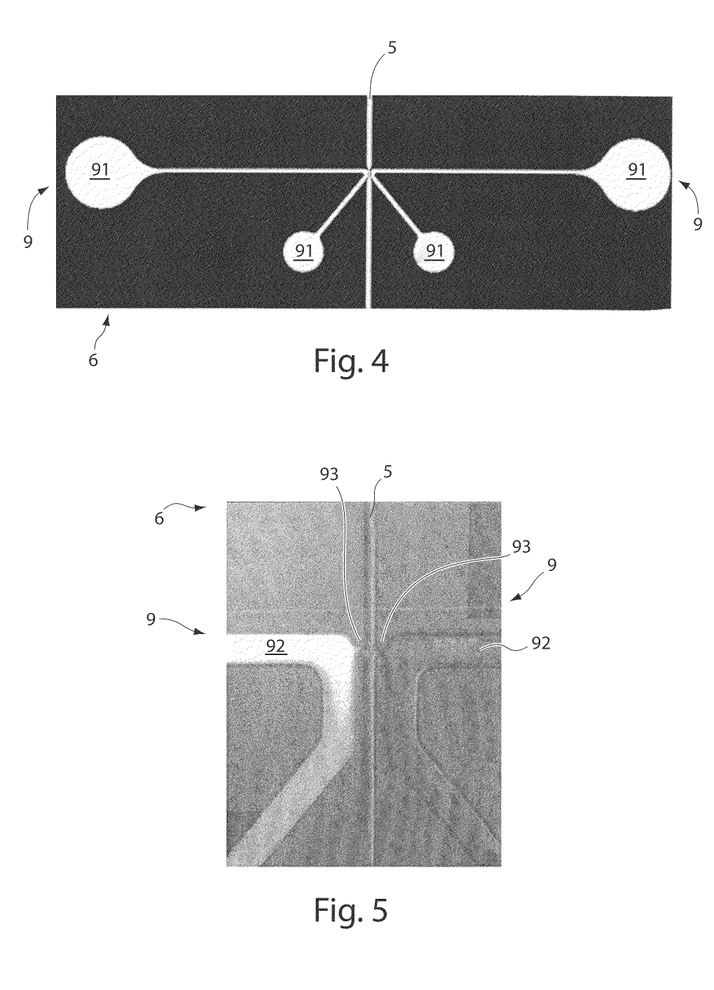

FIG. 4 shows a top view of a portion of a plate 6 or other substrate that includes a fluid channel 5, e.g., like the one described in the FIG. 1 embodiment above. The fluid channel 5 is shown extending from top to bottom in FIG. 4, and may be configured to conduct the flow of a liquid, e.g., a liquid including one or more cells 51 and/or other materials. A pair of electrode channels 9 are also shown, which each include a conductive material reservoir 91 at ends of the electrode channel 9 that are connected by an electrode portion 92. Although two conductive material reservoirs 91 are included with each electrode channel 9 in this embodiment, only one reservoir 91 may be included in other embodiments. In this embodiment, the pair of electrode channels 9 may include a conductive material, such as solder, in the electrode portion 92 so that an electrode is formed on opposite sides of the channel 5 at the location where the electrode portion 92 is adjacent the channel 5. In forming the electrode, solder or other suitable material may be provided in one of the reservoirs 91 (whether in liquid or solid form), and the liquid conductive material allowed to flow from the reservoir 91 and into the electrode portion 92. If a second reservoir 91 is provided, the conductive material may flow through the electrode portion 92 and into the second reservoir 91, ensuring complete electrode formation.

FIG. 5 shows a close up view of the electrode portion 92 of the FIG. 4 embodiment at a location where the electrode portion 92 is adjacent the channel 5. In this view, one of the electrode portions 92 (on the left side) has a conductive material (in this case solder) in the electrode portion 92 of the electrode channel 9. The right side electrode portion 92 in this view does not have conductive material positioned in it yet, but the passageway 93 is formed. In accordance with an aspect of the invention, a passageway 93 is formed between the electrode portion 92 and the channel 5 before the conductive material is allowed to flow into the electrode portion 92. However, the passageway 93 is arranged so that the liquid conductive material (e.g., melted solder) does not flow through the passageway 93 and into the channel 5, e.g., because the size or other feature of the passageway 93 prevents the liquid conductive material from flowing. For example, the passageway 93 may be sized so that surface tension at the surface of the liquid conductive material prevents the material from flowing into the passageway 93. The result is that an electrode may be formed in fluid and electrical communication with the channel 5 via the passageway 93, with little or no risk of having the electrode material flow into the channel 5 when the electrode is formed. In this embodiment, the electrode portion 92 has a size of about 60 microns by about 15 microns, and the passageway 93 has a size of about 10 microns by about 15 microns, but other sizes and configurations are possible.

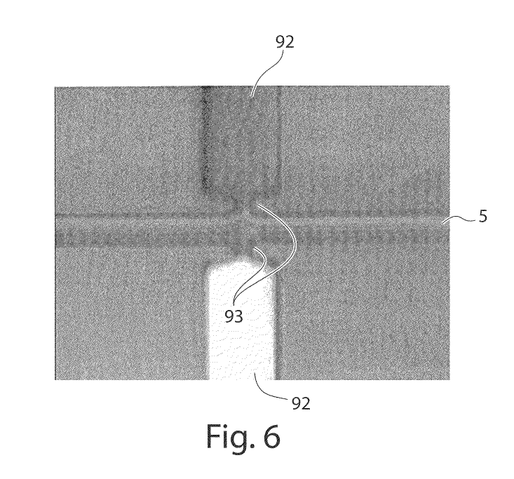

Although in the embodiment above, the electrode portion 92 is arranged so that the electrode portion 92 extends from a conductive material reservoir 91 toward the fluid channel in a direction transverse to the flow path of the channel 5 to a location where the electrode channel is adjacent the fluid channel, and then extends away from the fluid channel, the electrode portion 92 may be arranged in other ways. For example, FIG. 6 shows an embodiment in which an electrode portion 92 extends transversely to a channel 5 and terminates at a location adjacent the channel 5. (In this view, the lower electrode portion 92 includes a conductive material, whereas the upper electrode portion 92 does not.)

### POSSIBLE ADVANTAGES AND APPLICATIONS OF EMBODIMENTS IN ACCORDANCE WITH ASPECTS OF THE INVENTION

High throughput quantitative single cell microinjection can be employed in at least the following areas, opening new possibilities and frontiers:

Genomics

Gene therapy involving the insertion of genes into cells to treat diseases. Embodiments in accordance with aspects of the invention may provide a fast and effective way to deliver genes inside the cells, and could enable certain types of gene therapy, like therapy for blood diseases (such as leukemia) and dendritic cell based immunotherapy (to treat cancer).

DNA delivery into cells for transfection of “difficult” cell lines

DNA delivery into cells for transfection of very large DNA molecules (potentially also entire chromosomes)

Delivery into cells of known amounts of a gene construct to study the expression level of a gene of interest in different conditions (change sequences in the promoter and see how this affect gene expression in vivo)

Delivery of known amounts of DNA sequences together with known amounts of enzymes that enhance DNA recombination in order to achieve easier/more efficient stable transfection, homologues recombination and site specific mutagenesis

RNA and RNA Interference (RNAi)

Delivery of known amounts of RNA for more efficient/easier RNAi (Microinjection based RNAi)

Delivery of RNA into cells for RNA silencing without the need of liposomes (treating cells with liposomes change their membrane composition, alters the activity of calcium dependent signaling cascades and introduces a number of biases in gene expression experiments)

Efficient delivery of known amounts of RNA constructs for RNA interference into cells in order to reduce the amount of constructs used in each experiments (RNA constructs used for RNA interference are very expensive).

Delivery of known amounts of RNA molecules together with known amounts of Dicer molecules to achieve standardized, efficient, RNAi across multiple cell lines and in different conditions

Delivery of known amounts of mRNA into cells to study some aspects of gene expression regulations at the posttranscriptional level (at present this kind of studies are either impossible or extremely difficult)

Delivery of known amounts of labeled RNA to study in vivo the half life of RNAs

Proteomics

Proteomics, the study of cellular protein function is currently held back by the difficulty of directly delivering proteins into living cells. Current methods make it difficult to study protein kinetics, localization, interactions, and expression without killing the cells or genetically modifying them and risking the production of artifacts.

Delivery of known amounts of labeled proteins to study their half life in vivo

Delivery of labeled proteins to perform in vivo studies of protein localization

Delivery of known amounts of proteins to study their effect in vivo without the need of over expressing proteins (Over expression of a protein doesn't give information about how much protein is expressed in the cell. When overexpressing proteins, its impossible to make titrations and therefore results are often qualitative)

Delivery of known amounts of tagged proteins in order to study their interactions with other proteins in vivo without the need of over expressing them.

Delivery of labeled antibodies into living cells for in vivo immunostaining and in vivo fluorescence-based western blotting

Delivery of nanoparticles across cell membranes

Drug Discovery

Delivery across the cell membrane of known amounts of drugs. This application would be extremely useful for drug discovery and development

Therapy

Intracellular delivery of drugs to specific subset of circulating blood cells

Cells Cryopreservation

High throughput microinjection of sugars into cells to improve cryopreservation of cells, especially oocytes

Stem Cells and Transgenic Organism

Delivery of DNA and/or DNA+recombination enzymes into embryonic stem cells for the development of transgenic stem cell lines

Delivery of DNA and/or DNA+recombination enzymes into zygotes for the development of transgenic organisms

Crystallization in Microfluidic Systems

Crystallization is a difficult process that is achieved after multiple trials in various crystallization conditions and is highly dependent on the reaction conditions. Currently, the low throughput of the crystallization condition screens and the difficulty in tightly controlling the crystallization conditions are holding back the field. Moreover, current crystallization protocols make use of large volumes of reagents. With certain embodiments of the invention, it is possible to deliver picoliter-sized droplets of one solution into another solution. For example, to perform crystallization by injecting the droplet into an antisolvent or by injecting a warm droplet into a cooled liquid to initiate crystallization.

Chemistry/Chemical Engineering

Microparticles fabrication

Pico and sub-pico droplet generation

While aspects of the invention has been described with reference to various illustrative embodiments, the invention is not limited to the embodiments described. Thus, it is evident that many alternatives, modifications, and variations of the embodiments described will be apparent to those skilled in the art. Accordingly, embodiments of the invention as set forth herein are intended to be illustrative, not limiting. Various changes may be made without departing from the invention.

1. A microfluidic injection device, comprising:
a closed microfluidic channel constructed and arranged to carry a material along a first path;
a nozzle constructed and arranged to emit a jet or droplet of fluid from an outlet of the nozzle and into the closed channel in an emission direction;
a reservoir for holding liquid and in fluid communication with the nozzle; and
a pressure generator adapted to create a pressure gradient in the reservoir to cause the nozzle to emit the jet or droplet of liquid from the outlet.

2. The device of claim 1, wherein the pressure generator creates a pressure wave in the reservoir that initially moves in a direction transverse to the emission direction.

3. The device of claim 1, further comprising:
a flexible membrane positioned between the pressure generator and the reservoir; and
wherein the pressure generator includes a piezoelectric element.

4. The device of claim 1, wherein the device is arranged to emit a jet from the nozzle with a speed of about 0 m/sec to about 40 m/sec, and the nozzle has a diameter of less than 20 microns.

5. The device of claim 1, further comprising a detector associated with the channel that is arranged to detect the presence of a target in the channel.

6. The device of claim 1, wherein the device is arranged to produce a jet of liquid from the nozzle so as to introduce the liquid through a cell membrane and into a cell interior, such that introduction of the liquid into the cell interior is accomplished so as to avoid damage to the cell membrane that would cause cell death.

7. The device of claim 1, wherein the device is arranged to produce a jet or droplet of liquid suitable for micro/nano particle synthesis or crystallization.

8. The device of claim 1, wherein the device is arranged to produce a jet of liquid from the nozzle so as to move the liquid toward a cell located in the channel, such that material present in the liquid impacts the cell membrane without piercing the cell membrane.

9. The device of claim 8, wherein the material in the liquid includes one or more of a particle, liposomes, tensioactives, chemicals, a dye or antibodies.

10. A method of introducing material into a cell, comprising:
providing a cell at a position adjacent an outlet of a nozzle, the cell having a cell membrane and a cell interior surrounded by the cell membrane;
providing a reservoir containing a fluid and in fluid communication with the nozzle;
producing a pressure gradient in the reservoir to urge fluid in the reservoir to move toward the nozzle; and
producing a jet of liquid, including the material, from the nozzle so as to pierce the cell membrane and introduce the liquid including the material through the cell membrane and into the cell interior, introduction of the liquid into the cell interior being accomplished so as to avoid damage to the cell membrane that would cause cell death.

11. The method of claim 10, further comprising:
producing a jet or droplet of liquid from the nozzle such that material present in the liquid impacts the cell membrane without piercing the cell membrane.

12. The method of claim 10, wherein the step of producing a pressure gradient comprises operating a piezoelectric element so as to move fluid in the reservoir.

13. The method of claim 10, wherein the material includes liposomes, tensioactives, chemicals, particles, chemicals, a dye or antibodies.

14. The method of claim 10, wherein the jet of liquid produced from the nozzle has a speed of about 0 m/sec to about 40 m/sec.

15. The method of claim 10, wherein an amount of liquid introduced into the cell interior has a volume of about a femtoliter to several picoliters.

16. The method of claim 10, wherein the step of providing a cell includes moving the cell along a channel that is in fluid communication with the nozzle; wherein the jet of liquid is produced and the cell membrane is pierced as the cell is moving along the channel.

17. A fluid injection device, comprising:
a channel constructed and arranged to carry a cell along a first path, the cell having a cell membrane and a cell interior;
a nozzle constructed and arranged to emit a jet or droplet of liquid from an outlet and into the channel in an emission direction;
a reservoir for holding liquid and in fluid communication with the nozzle; and
a pressure generator adapted to create a pressure gradient in the reservoir to cause the nozzle to emit the jet or droplet of liquid from the outlet;
wherein the jet or droplet of liquid is emitted so as to pierce the cell membrane and introduce the liquid through the cell membrane and into the cell interior, the introduction of the liquid into the cell interior being accomplished so as to avoid damage to the cell membrane that would cause cell death.

18. The device of claim 17, wherein the pressure generator creates a pressure wave in the reservoir that initially moves in a direction transverse to the emission direction.

19. The device of claim 17, further comprising:
a flexible membrane positioned between the pressure generator and the reservoir.

20. The device of claim 17, wherein the pressure generator includes a piezoelectric element.

21. The device of claim 17, wherein the device is arranged to emit a jet from the nozzle with a speed of about 0 m/sec to about 40 m/sec.

22. The device of claim 17, wherein an amount of liquid introduced into the cell interior has a volume of about a femtoliter to several picoliters.

23. The device of claim 17, wherein the device is arranged to produce a jet of liquid from the nozzle so as to accelerate the liquid toward a cell, such that material present in the liquid impacts the cell membrane without piercing the cell membrane.

24. The device of claim 25, wherein the jet or droplet of liquid includes liposomes, tensioactives, chemicals, particles, chemicals, a dye or antibodies.
